(19) United States
(12) Patent Application Publication
(10) Pub. No.: US 2024/0390598 A1
(43) Pub. Date: Nov. 28, 2024

(54) CHECKING A SENSOR OF A DRUG DELIVERY DEVICE OR OF A DRUG DELIVERY ADD-ON DEVICE

(71) Applicant: Sanofi, PARIS (FR)

(72) Inventors: Jonathan Jason Drake, Warwick (GB); Paul Richard Draper, Warwick (GB); Anthony Paul Morris, Warwick (GB); Aidan Michael O`Hare, Warwick (GB); Ronald Antony Smith, Salir, Loule (PT); John Whitehouse, Harvington, Evesham (GB)

(21) Appl. No.: 18/694,109

(22) PCT Filed: Sep. 22, 2022
(86) PCT No.: PCT/EP2022/076297

§ 371 Date: Mar. 21, 2024

(30) Foreign Application Priority Data
Sep. 24, 2021 (EP) 21315177.2

(51) Int. Cl. A61M 5/315

(52) U.S. Cl. CPC A61M 5/3157; A61M 5/31583; A61M 2205/3306; A61M 2205/702

(57) ABSTRACT

A method for checking a sensor of a drug delivery device or of a drug delivery add-on device is disclosed, wherein the sensor is provided and configured to detect expelling of a drug dose being delivered with the drug delivery device and to output a respective sensor signal, and wherein the method comprises taking readings of the sensor signal in addition to readings taken for calculating the dose delivered with the drug delivery device, and processing the additionally taken readings for determining at least one condition of the sensor.

### FIELD

The present disclosure relates to checking a sensor of a drug delivery device or of a drug delivery add-on device, particularly for errors and/or faults and/or degradation of the sensor.

### BACKGROUND

WO2016131713A1 relates to a data collection device for attachment to an injection device and collecting medicament dosage information therefrom. The data collection device may comprise a mating arrangement configured for attachment to the injection device, a sensor arrangement configured to detect movement of a movable dosage programming component of the injection device relative to the data collection device during delivery of a medicament, and a processor arrangement configured to, based on said detected movement, determine a medicament dosage administered by the injection device. The sensor arrangement may include an optical sensor, for example, an optical encoder unit, particularly including a light source, such as a light emitting diode (LED) and a light detector, such as an optical transducer. The processor arrangement may be configured to monitor a time period elapsed since a pulse was output by the optical encoder and to determine said medicament dosage if said time period exceeds a predetermined threshold.

WO2019101962A1 relates to medicament injection devices. An injection device may comprise a movable dosage programming component comprising a rotary encoder system having a predefined angular periodicity, a sensor arrangement comprising a first optical sensor configured to detect movement of the movable dosage programming component relative to the sensor arrangement during dosing of a medicament, wherein the first optical sensor is configured to operate in a strobe-sampling mode at a first frequency, and a second optical sensor configured to detect movement of the rotary encoder system relative to the second optical sensor, wherein the second optical sensor is configured to operate in a strobe-sampling mode at a second frequency lower than the first frequency, and a processor arrangement configured to, based on said detected movement, determine a medicament dosage administered by the injection device. A controller may be provided to control a sensor arrangement comprising the optical sensors, for example infrared (IR) reflective sensors, which emit IR light from an LED and detect IR light reflected from IR reflective regions of the encoder system.

### SUMMARY

This disclosure describes methods and devices for checking a sensor of a drug delivery device or of a drug delivery add-on device, particularly for errors and/or faults and/or degradation of the sensor.

In one aspect the present disclosure provides a method for checking a sensor of a drug delivery device or of a drug delivery add-on device, wherein the sensor is provided and configured to detect expelling of a drug dose being delivered with the drug delivery device and to output a respective sensor signal, and wherein the method comprises taking readings of the sensor signal in addition to readings taken for calculating the dose delivered with the drug delivery device, and processing the additionally taken readings for determining at least one condition of the sensor. Particularly, the additionally taken readings are readings, which are neither provided nor used for calculating the dose delivered with the drug delivery device. The additionally taken readings may be regarded as dedicated sensor condition determination readings in contrast to dose delivery calculation readings. Therefore, the additionally taken readings may increase the overall readings taken from the sensor signal. The additionally taken readings allow to check the sensor also when dose is delivered with the drug delivery device. Thus, sensor checks may be performed when a drug dose is to be delivered. Moreover, the provision of the additionally taken readings offer more flexibility with regard to determining a sensor condition since the additional readings may differ from the readings used for calculating the delivered dose. Thus, the additionally taken readings may allow for example to perform different check of a sensor to determine its condition in contrast to rely a sensor check on readings taken for calculating a delivered dose since these later readings usually have parameters predetermined for the delivered dose calculation. The method is particularly suitable for application with analogue sensors, which may suffer from degradation, faults, and/or errors, for example accelerometers, light sensors, sound sensors, pressure sensors, temperature sensors, proximity sensors, infrared sensors, ultrasonic sensors, colour sensors, humidity sensors, tilt sensors, flow sensors, magnetic/Hall effect sensor, radiation sensors, lidar, electrical current sensors, optical sensors, force/torque sensors, strain gauges. The method may help to check for a repeatable performance of the sensor particularly over the lifetime of the drug delivery device or the drug delivery add-on device, in which the sensor is integrated. Due to the additionally taken readings, the method may be performed under normal operation of the drug delivery device or drug delivery add-on device. Particularly, the method may be performed with any standard sensor operation, i.e. when the sensor is in normal operation for detecting a dose delivery, at certain intervals, for example every n^{th} standard operation (n=1,2,3, . . . ), upon occurrence of certain events such as at certain times, dates, and/or upon receipt of an external command, for example from an external device or due to an user input.

The term “condition of the sensor” as used herein particularly may mean any operating condition of the sensor, for example accuracy of the sensor signal with regard to a sensed parameter, errors of the sensor signal, and/or faults of the sensor signal. For example, “condition of the sensor” may comprise a failure of the sensor, i.e. when the sensor signal is completely faulty, a degradation of the sensor, i.e. when the sensor 20) signal does not correctly represent sensed parameters due to the degradation, and/or errors such as electrical failures in circuitry of the sensor, for example electrical failures in resistors, capacitors, inductors, tracks of circuitry connected to the sensor, or the sensor itself resulting in for example glitches or spikes of the error signal or an entirely erroneous error signal, such as an error signal permanently stuck on one level due to a short-circuit.

The term “readings of the sensor signal” as used herein particularly comprises samples taken from an analogue sensor signal at certain times. A “reading” may be taken over a specific time interval, which may vary. For example, a long “reading” may be taken over a time interval, which is longer than a normal or a short “reading”. A “reading” may also comprise several “sub” readings, i.e. a reading is not limited to a single reading. For taking readings, a sample and hold circuitry may be used, comprising a sampling switch and a capacitor for storing a taken sample. Taken readings may be digitized for further processing, such as the determining of the at least condition of a sensor. However, also an analogue processing is possible, for example by comparing taken readings with predefined thresholds with comparator circuitries. The term “readings of the sensor signal” as used herein may also comprise a driving of an “emitting” part of the sensor for a variable amount of time, but that the reading could still be the same. In embodiments with optical sensors, for example a long “reading” may mean driving an emitter such as a LED (Light Emitting Diode) for a longer period before taking a reading from the detector side of the sensor. For both short and long pulses the effective time the reading is taken over may be the same time; it may be just that more charge has built up on the detector because it was exposed to a longer pulse of light.

In embodiments, the additionally taken readings may be taken before, after, at the start of, at the end of, and/or during the delivery of a drug dose with the drug delivery device, and/or the additional readings are taken over the entirety, a portion, or multiple portions of the delivery of a drug dose with the drug delivery device. For example, when a user begins to select a dose with the drug delivery device, an electronic system for detecting the selected dosage may be activated and switch on the sensor to generate an output signal corresponding to the dose selecting. While the output signal is available, a sequence of readings may be taken. Also, for example, additional readings may be taken when drug delivery ended, but the sensor still generates a sensor signal.

In further embodiments, the additional readings may be taken with the same parameters as the readings for calculating the delivered dose. Thus, no change of the taking of readings is required for taking the additional readings, and any control of the readings taking may not be configured to change any readings parameters. For example, the duration of taking a reading, the frequency of taking readings, the level of taking readings may remain unchanged for the readings for calculating the delivered dose and the additional readings, which may reduce the efforts for implementing the reading control.

In alternative embodiments, the additional readings may be taken with different parameters than the readings for calculating the delivered dose, particularly the additional readings are taken for a longer or a shorter time, at a higher or lower level and/or frequency than the time, frequency or level for or at which the readings for calculating the delivered dose are taken. This particularly allows to adapt the taking of the additional readings to check requirements, for example the additional readings may be taken with a higher frequency than the normal readings for delivered dose calculation, which may increase the check accuracy.

In yet further embodiments, at least one period of not taking readings may be included between the taking of the readings for calculating the delivered dose and the taking of the additional readings. Such a “non-measurement” period may account for debouncing or other transitory effects such as electric charge dissipations in electric devices of the sensor circuitry and/or control and processing circuitry, and, may, therefore, help to improve the overall performance of the method.

In embodiments, the processing of the additionally taken readings for determining a condition of the sensor may comprise examining the additionally taken readings against least one threshold and determining the at least one condition of the sensor depending on the examination. This may allow to distinguish potential error, fault or degradation conditions of the sensor. For example different thresholds may be provided for different conditions, such as at least one error threshold, fault threshold, and degradation threshold. Additionally taken readings may then be compared to the at least one thresholds, and depending on the comparison, one or more conditions of the sensor may be determined.

In an embodiment, the method may further comprise examining the readings taken for calculating the delivered dose against the at least one threshold and using this further examination for determining the at least one condition of the sensor. The further examination based on the readings taken for delivered dose calculation may be either independently from the examination based on the additionally taken readings, or together with the latter. It may be for example used for a plausibility check of the at least one sensor condition determined based on the additionally taken readings.

In embodiments, the method may further comprise storing the at least one determined condition of the sensor in a dataset together with the calculated delivered dose, or storing the at least one determined condition of the sensor in a dataset separate from the calculated delivered dose. The storing together with the calculated delivered dose has the advantage that for example an external device used for processing the delivered dose after receipt from the drug delivery device or drug delivery add-on device may immediately see the sensor condition(s) and for example assess the accuracy of the calculated delivered dose. The storage of the determined sensor condition(s) in a separate dataset reduces the amount of data to be transmitted to an external device for processing. The separate dataset may be for example in a dedicated memory area of for example an internal memory of a processor, particularly a controller provided and configured for controlling the sensor and may be accessible for example for the purpose of service to check whether the drug delivery device or drug delivery add-on device still delivers accurate measurement results.

In embodiments, the method may further comprise generating an error signal when the at least one determined condition of the sensor does not fulfil one or more predefined conditions. The error signal may be immediately processed, for example for stopping usage of the drug delivery device or add-on device due to sensor degradation, sensor errors, and/or a sensor failure, particularly when the accuracy of delivered dose calculation can no longer be ensured to a predefined degree.

In an embodiment, the method may further comprise outputting an alert informing of the non-fulfilment of the one or more predefined conditions by the at least one determined condition of the sensor. The alert may be for example a visible, tactile and/or audible alert, such as a blinking light signal, a vibration and/or a buzzer sound, particularly generated by a LED (Light Emitting Diode), a vibration motor, a buzzer or loudspeaker, for example integrated in the drug delivery device or add-on device and/or an external device connected to the drug delivery device or add-on device and receiving the alert.

In embodiments, the sensor may comprise an optical sensor having a light emitter and light receiver and being provided and configured for detecting transitions between different regions of a moving component of the drug delivery device and to output a signal as the sensor signal comprising the detected transitions, wherein the optical sensor is controlled with parameters for taking the additional readings differing from the parameter for taking the readings for calculating the dose delivered with the drug delivery device. For example, the optical sensor may be part of a dose selection and expelling mechanism such as described in WO2019101962A1. The method may for example control the optical sensor with another control signal for generating a longer light emission for taking the additional readings than the light emission for taking readings for dose calculation, and/or it may control the optical sensor with a control signal to switch off light emission when taking an additional reading.

In a further aspect the present disclosure provides a device for controlling a sensor of a drug delivery device or a drug delivery add-on device, the device being configured to implement a method as disclosed herein, the device particularly comprising a controller, particularly a microcontroller, the controller being configured by a program to implement the method as disclosed herein. The program may be for example part of a firmware of a controller of an electronic system of a drug delivery device or add-on device, which is provided to implement dose delivery calculation.

In embodiments, the device may further comprise one or more of the following: a storage unit for storing calculated delivered doses and/or at least one determined condition of the sensor; a communication unit configured for communicating with an external computing device; a user interface configured for receiving user inputs for configuring the device and/or for outputting information about delivered doses and/or at least one determined condition of the sensor; a display unit configured for displaying information about delivered doses and/or at least one determined condition of the sensor. Particularly, the device may comprise an electronic system comprising one or more of the before listed devices. The electronic system may for example implement by means of the communication unit a connectivity of the drug delivery device or add-on device with external computing devices for data processing, such as laptop computers, desktop computers, cloud computers, handheld computers such as smartphones, tablet computers, smartwatches, server computers, computers provided and configured for medical purposes. The electronic system may also implement the user interface and the display unit by means of a touch screen.

In a yet further aspect the present disclosure provides a sensor unit of a drug delivery device or of a drug delivery add-on device, wherein the sensor unit comprises a sensor controlled by a device as disclosed herein, and wherein the sensor unit is provided and configured for integration in a drug delivery device or a drug delivery add-on device. The sensor unit may for example comprise a printed circuit board (PCB) with the electronic system comprising the controller and further electronic components required for operation of the controller and the at least one sensor, and the at least one sensor may be wired with the PCB.

In a still further aspect the present disclosure provides a drug delivery device or a drug delivery add-on device, particularly an injection pen, comprising a sensor unit as disclosed herein.

### BRIEF DESCRIPTION OF THE FIGURES

FIG. 1 shows an injection device according to a first embodiment;

FIG. 2 is an elevated side view of a first type of encoder system;

FIG. 3 is a plan view of the encoder system shown in FIG. 2;

FIG. 4 is an elevated side view of a second type of encoder system;

FIG. 5 is a plan view of the encoder system shown in FIG. 4;

FIG. 6 shows a schematic block diagram of an embodiment of a device controller;

FIG. 7 shows an example waveform of a sensor signal generated by a sensor and readings taken from this sensor signal; and

FIG. 8 shows a further waveform of a sensor signal generated by a sensor such and readings taken from this sensor signal.

### DETAILED DESCRIPTION OF SOME EMBODIMENTS

In the following, embodiments of the present disclosure will be described with reference to injection devices, particularly an injection device in the form of a pen. The present disclosure is however not limited to such application and may equally well be deployed with other types of drug delivery devices, particularly with another shape than a pen. All absolute values are herein shown by way of example only and should not be construed as limiting.

An example of an injection pen where an injection button and grip are combined and its mechanical construction is described in detail in the international patent application WO2014033195A1. Another example of an injection where there are separate injection button and grip components is described in WO2004078239A1.

In the following discussion, the terms “distal”, “distally” and “distal end” refer to the end of an injection pen towards which a needle is provided. The terms “proximal”, “proximally” and “proximal end” refer to the opposite end of the injection device towards which an injection button or dosage knob is provided.

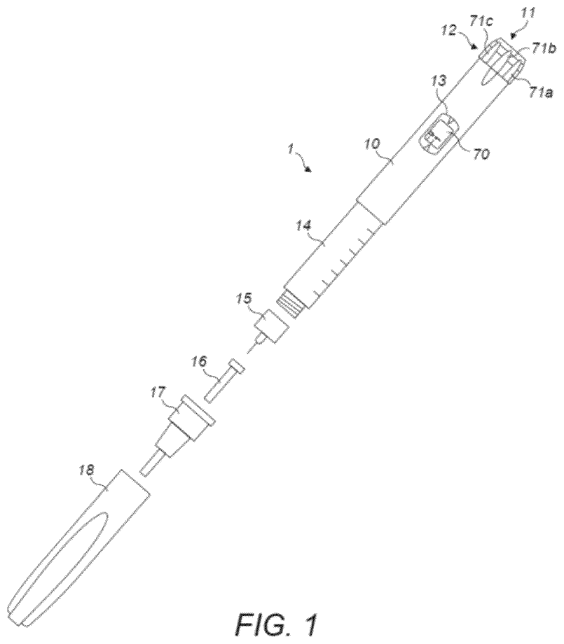

FIG. 1 is an exploded view of an injection pen 1. The injection pen 1 of FIG. 1 is a pre-filled, disposable injection pen that comprises a housing 10 and contains an insulin container 14, to which a needle 15 can be affixed. The needle is protected by an inner needle cap 16 and either an outer needle cap 17 other cap 18. An insulin dose to be ejected from injection pen 1 can be programmed, or ‘dialled in’ by turning a dosage knob 12, and a currently programmed dose is then displayed via dosage window 13, for instance in multiples of units. For example, where the injection pen 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming. In this example, the dosage knob 12 includes one or more formations 71a, 71b, 71c to facilitate attachment of a data collection device (drug delivery or injection add-on device).

The injection pen 1 may be configured so that turning the dosage knob 12 causes a mechanical click sound to provide acoustical feedback to a user. The dial sleeve 70 mechanically inter-acts with a piston in insulin container 14. In this embodiment, the dosage knob 12 also acts as an injection button. When needle 15 is stuck into a skin portion of a patient, and then dosage knob 12 is pushed in an axial direction, the insulin dose displayed in display window 13 will be ejected from injection pen 1. When the needle 15 of injection pen 1 remains for a certain time in the skin portion after the dosage knob 12 is pushed, a high percentage of the dose is actually injected into the patient's body. Ejection of the insulin dose may also cause a mechanical click sound, which is however different from the sounds produced when rotating the dosage knob 12 during dialling of the dose.

In this embodiment, during delivery of the insulin dose, the dosage knob 12 is returned to its initial position in an axial movement, without rotation, while the dial sleeve 70 is rotated to return to its initial position, e.g. to display a dose of zero units.

Injection pen 1 may be used for several injection processes until either the insulin container 14 is empty or the expiration date of the medicament in the injection pen 1 (e.g. 28 days after the first use) is reached.

Furthermore, before using injection pen 1 for the first time, it may be necessary to perform a so-called “prime shot” to remove air from insulin container 14 and needle 15, for instance by selecting two units of insulin and pressing dosage knob 12 while holding injection pen 1 with the needle 15 upwards. For simplicity of presentation, in the following, it will be assumed that the ejected amounts substantially correspond to the injected doses, so that, for instance the amount of medicament ejected from the injection pen 1 is equal to the dose received by the user. Nevertheless, differences (e.g. losses) between the ejected amounts and the injected doses may need to be taken into account.

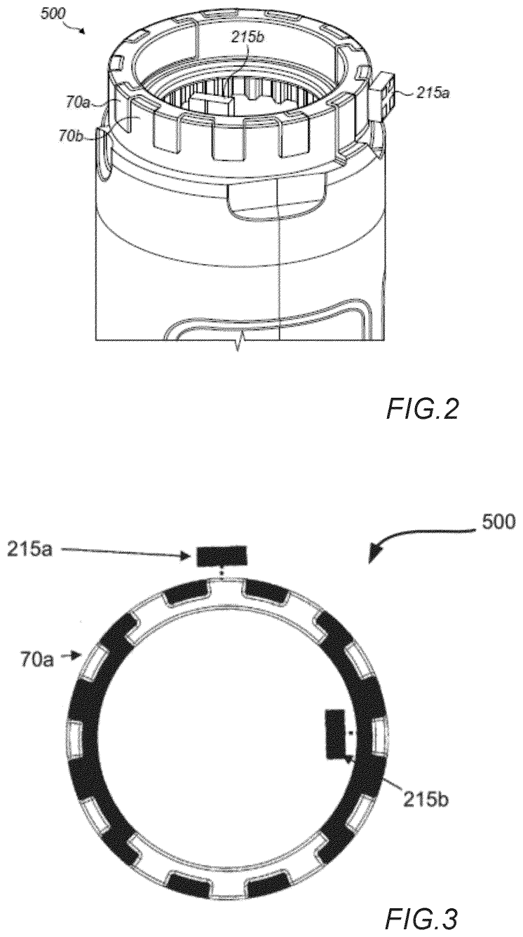

As explained above, the dosage knob 12 also functions as an injection button so that the same component is used for dialling and dispensing. A sensor arrangement 215 (FIGS. 2 and 3) comprising one or more optical sensors may be mounted in the injection button or dosage knob 12 which is configured to sense the relative rotational position of the dial sleeve 70 relative to the injection button 12. This relative rotation can be equated to the size of the dose dispensed or delivered and used for the purpose of generating and storing or displaying dose history information. The sensor arrangement 215 may comprise a primary (optical) sensor 215a and a secondary (optical) sensor 215b. This sensor arrangement is only an example embodiment, and other, different sensor arrangements may be used. For the sake of simplicity, in the following the embodiment with only the sensor arrangement 215 is described in detail, but it should be noted that also other sensor arrangements can be applied, for example arrangement with a single sensor or more than two sensors, arrangements with several different and/or equal sensors pointing at the same and/or different sets of reflective areas. Also, sensor arrangements without reflective areas are possible, for example when a rotatable encoder with alternating translucent and opaque areas is positioned between an emitter and a sensor of the sensor arrangement so that radiartion emitted by the emitter can pass the rotable encoder only when a translucent area is between the emitter and the sensor. The sensor arrangement 215 may be also mounted in drug delivery or injection add-on device, which may be provided for usage with different injection devices 1 and configured to collect data acquired with the sensor arrangement 215.

) The optical sensors 215a, 215b of the sensor arrangement 215 may be employed with an encoder system, such as the systems 500 and 900 shown in FIGS. 2, 3 and 4, 5, respectively. The encoder system is configured for use with the device 1 described above.

As shown in FIG. 2 and FIG. 3, the primary sensor 215a and secondary sensor 215b are configured to target specially adapted regions at the proximal end of the dial sleeve 70. In this embodiment, the primary sensor 215a and secondary sensor 215b are IR reflective sensors. Therefore, the specially adapted proximal regions of the dial sleeve 70 are divided into a reflective area 70a and a non-reflective (or absorbent) area 70b. The part of the dial sleeve 70 comprising the reflective area 70a and a non-reflective (or absorbent) area 70b may be termed an encoder ring.

To keep production costs to a minimum, it may be favourable to form these areas 70a, 70b from injection moulded polymer. In the case of polymer materials, the absorbency and reflectivity could be controlled with additives, for example carbon black for absorbency and titanium oxide for reflectivity. Alternative implementations are possible whereby the absorbent regions are moulded polymer material and the reflective regions are made from metal (either an additional metal component, or selective metallisation of segments of the polymer dial sleeve 70).

Having two sensors facilitates a power management technique described below. The primary sensor 215a is arranged to target a series of alternating reflective regions 70a and non-reflective regions 70b at a frequency commensurate with the resolution required for the dose history requirements applicable to a particular drug or dosing regime, for example, 1 IU. The secondary sensor 215b is arranged to target a series of alternating reflective regions 70a and non-reflective regions 70b at a reduced frequency compared to the primary sensor 215a. It should be understood that the encoder system 500 could function with only a primary sensor 215a to measure the dispensed dose.

The secondary sensor 215b facilitates the power management technique described below.

The two sets of encoded regions 70a, 70b are shown in FIGS. 2 and 3 concentrically with one external and the other internal. However, any suitable arrangement of the two encoded regions 70a, 70b is possible. Whilst the regions 70a, 70b are shown as castellated regions, it should be borne in mind that other shapes and configurations are possible.

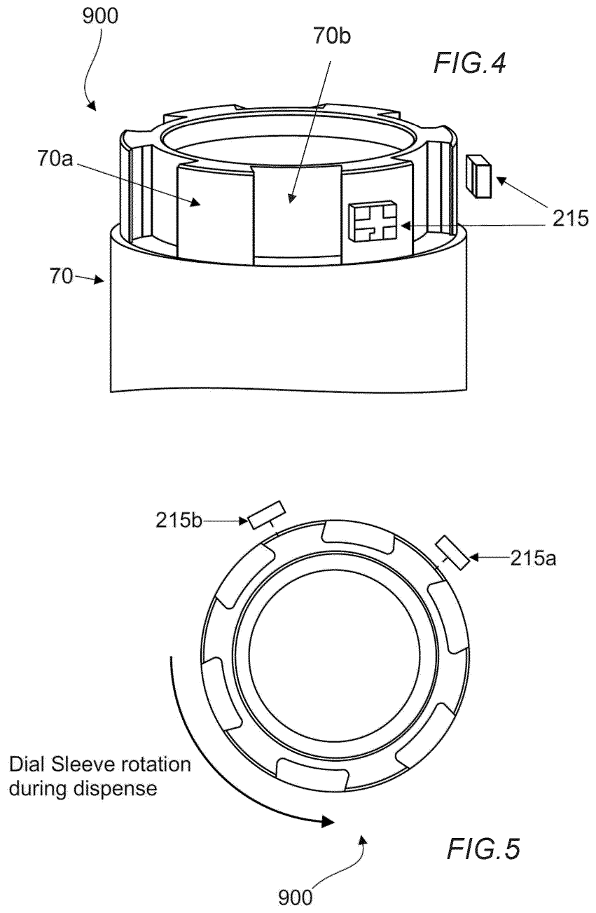

As shown in FIG. 4, the two sensors 215 from this embodiment are configured to target specially adapted regions 70a, 70b of the dial sleeve 70. In this embodiment IR reflective sensors are used, therefore the regions of the dial sleeve 70 are divided into reflective and absorbent segments 70a, 70b. The segments 70a, 70b may also be referred to herein as flags.

Unlike the encoder system 500 described above in relation to FIGS. 2 and 3, the encoder system 900 shown in FIGS. 4 and 5 has both IR sensors 215 target the same type of region 70a, 70b. In other words, the sensors 215 are arranged so that they both face reflective regions 70a or both face absorbent regions 70b at the same time. During the dispensing of a dose, the dial sleeve 70 rotates anti-clockwise 15° relative to the injection button 210 for every medicament unit that has been dispensed. The alternate flag elements are in 30° (or two unit) sections. The sensors 215 are arranged to be out of phase with each other, such that the angle between them equates to an odd number of units (e.g. 15°, 45°, 75°, etc.), as shown in FIG. 5.

The encoder system 900 shown in FIG. 5 has 12 units per revolution, i.e. 12 alternating regions 70a, 70b. In general, embodiments work with any multiple of 4 units per revolution. The angle, or, between sensors 215 can be expressed by the below equation, where both m and n are any integers and there are 4 m units dispensed per revolution.

$$α = ( 2 n - 1 ) \frac{360}{4 m} \\ Angle between sensors \qquad Equation$$

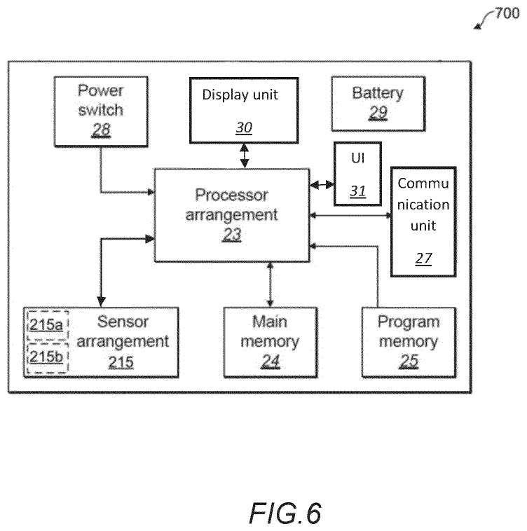

The device 1 or an add-on device for attachment to the device 1 may also include a sensor unit 700, as shown schematically in FIG. 6. The sensor unit 700 may comprise the sensor arrangement 215 including the two sensors 215a, 215b and a device for controlling the sensor arrangement 215. The controlling device may comprise a processor arrangement 23 including one or more processors, such as a microprocessor, a Digital Signal Processor (DSP), Application Specific Integrated Circuit (ASIC), Field Programmable Gate Array (FPGA) or the like, memory units 24, 25, including program memory 24 and main memory 25, which can store software for execution by the processor arrangement 23, a communication unit or output 27, which may be a wireless communications interface for communicating with another device via a wireless network such as Wi-Fi™ or Bluetooth®, and/or an interface for a wired communications link, such as a socket for receiving a Universal Series Bus (USB), mini-USB or micro-USB connector, a display unit 30, for example a LCD (Liquid Crystal Display), one or more LEDs, and/or an electronic paper display, a user interface (UI) 31, for example one or more buttons and/or touch input devices, a power switch 28, and a battery 29.

The controlling device components 23, 24, 25, 27, 28, 29, 30, 31 may be soldered on a PCB containing the wiring of components. The sensor arrangement 215 may be also attached to the PCB, or may be wired with the processor arrangement 23. The implementation of the sensor unit 700 depends on the drug delivery device or drug delivery add-on device, in which it should be integrated. For example, a PCB with the components 23, 24, 25, 27, 28, 29, 30, 31 may be integrated in the distal end of the injection device 1, and the sensors 215a, 215b may be arranged as shown in FIGS. 2 and 3 and connected to the PCB via wires. At least some of the components 23, 24, 25, 27 may be also comprised by a SoC (System on Chip) or microcontroller.

A firmware stored in the program memory 25 may configure the processor arrangement 23 to control the sensor arrangement 215 such that expelling of a drug dose being delivered with the device 1 can be detected and the sensors 215a, 215b each output a sensor signal corresponding to the detected delivered drug dose, particularly as described above with regard to the FIGS. 2 and 3. The processor arrangement 23 receives the sensor signal of each of the sensors 215a, 215b and takes readings of each sensor signal, which are processed to calculate the delivered dose. A reading may comprise for example one or more voltage samples of an analogue voltage signal of the sensor 215a, 215b. A reading may also comprise an integration of an analogue voltage signal of the sensor 215a, 215b over a certain time span. Instead of voltage signals, also electric currents, electric charges or another output signal generated by a sensor may be used for taking readings, for example frequencies of a sensor signal, frequency shifts. The readings may be taken by each sensor 215a, 215b during operation of the injection device 1 to measure the number of units dispensed by the device 1. The measuring of the number of dispensed units ma comprise counting peaks of each sensor signal and deriving from the counted peaks the delivered dose as described below by in more detail.

It is advantageous to be able to minimise the power usage of the encoder system 500 so that the size of a battery 29 needed to be packaged into the device 1 can be minimised. The sensors 215a, 215b used in this embodiment require a certain amount of power to operate. This embodiment is arranged such that the sensors 215a, 215b can be switched on and off intermittently at a controlled frequency (i.e. in a strobe-sampling mode). There is inherently a limit to the maximum rotational speed that can be counted by a sampled encoder system before aliasing occurs. Aliasing is the phenomenon where the sampling rate is less than the rate at which sensed regions pass the sensor which means that a miscount could occur when a region change is missed. The secondary sensor 215b with a reduced frequency compared to the primary frequency 215a can tolerate a higher rotational speed before it too becomes aliased. Whilst the secondary sensor 215b is not able to resolve the dose dispensed to the same resolution as the primary sensor 215a, the output of the secondary sensor 215b remains reliable at higher speeds. Therefore both sensors 215a, 215b are used in combination to be able to accurately determine dose delivered up to a first threshold rotational (dispensing) speed. The sensors 215a, 215b can then be used to determine an approximate dose delivered up to a second (higher) threshold dosing speed. At speeds above the second threshold speed the sensors 215a, 215b will not be able to accurately or approximately determine the dose delivered, therefore the second threshold is set above a speed which is not physically possible in the injection pen 1.

The first speed threshold is determined by the sampling rate of primary sensor 215a and the frequency of encoder region transitions, which is fixed at the resolution required by the intended drug or dosing regime (for example one transition per 1 IU). The second speed threshold is determined by the sampling rate of the secondary sensor 215b and the frequency of encoder region transitions. The first threshold is set such that the largest range of dispensing speeds can be covered by the system for accurate reporting of dose dispensed.

The example embodiment shown in FIG. 3 has primary sensor 215a targeting region transitions at 1 transition per 1 IU of dose delivered and the secondary sensor 215b targeting region transitions at 1 transition per 6 IU of dose delivered. Other options are possible which include 1 transition per 2 IU, 1 transition per 4 IU, 1 transition per 8 IU and 1 transition per IU units. These options are each possible because there are 24 separate regions 70a, 70b per revolution in the encoder system 500 shown in FIG. 3. In general, if the number of separate regions 70a, 70b per revolution were n units then there would be options at one region transition per m units where m was any integer factor of n greater than 1 and less than n.

The slower the sampling frequency of both sensors 215a, 215b, the lower the power consumption required and therefore the smaller the required size of the battery 29. It is therefore optimal to minimise, by design, the sampling frequency as far as is practical.

The firmware stored in the program memory 25 and being executed by the processor arrangement 23 for detecting the delivered dose also implements a method for checking the sensors 215a, 215b of the sensor arrangement 215 as described in the following in detail. The checking method configures the processor arrangement 23 to take readings of the signal of at least of the sensors 215a, 215b in addition to the readings taken for calculating the delivered dose.

The additional readings for sensor checking may be taken before, after, at the start of, at the end of, and/or during the delivery of a drug dose with the drug delivery device. For example, when a dose is selected with the drug delivery device, readings may be taken of signals of the sensors 215a, 215b and processed for checking the sensors 215a, 215b. Also, during drug delivery, additional readings may be taken for checking the sensors 215a, 215b. In such case, the additional readings may be taken time-shifted to the readings taken for calculating the expelled dose. Yet further, the additional readings may be taken at the start of and/or at the end of expelling a selected drug dose, for example triggered by pushing the dosage knob 12 in an axial direction to eject a selected drug dose, wherein the sensors 215a, 215b generated sensor signals, for example when the encoder system 500 rotates during expelling of the drug dose. The additional readings may be taken over the entirety, a portion, or multiple portions the delivery of a drug dose with the drug delivery device. The number of additionally taken readings may influence the check result. For example, a large number of additionally taken readings may result in a more averaged check result.

The additional readings could be the same as the standard readings taken for dose calculation, or different, in order to perform tests, particularly self-check tests. Any additional readings may be taken over a longer or shorter time span than a standard reading by one or more sensors, and, depending on the sensor technology, be at a higher or lower level and/or frequency than a standard reading. In the example of an optical sensor such as the sensors 215a, 215b (FIGS. 2, 3), a long reading at maximum brightness could be performed, followed by a long reading at minimum brightness. Periods of non-measurement may be included before or between readings in order to account for debouncing or other transitory effects such as charge dissipation.

By processing the additionally taken reading, particularly by examining the results of the one or more additional readings against one or more threshold values, it is possible to distinguish potential error, fault or degradation conditions of the sensor, which output the signal from which the additional readings were taken. Additionally, the standard readings may be examined, either alone or in conjunction with the self-check readings.

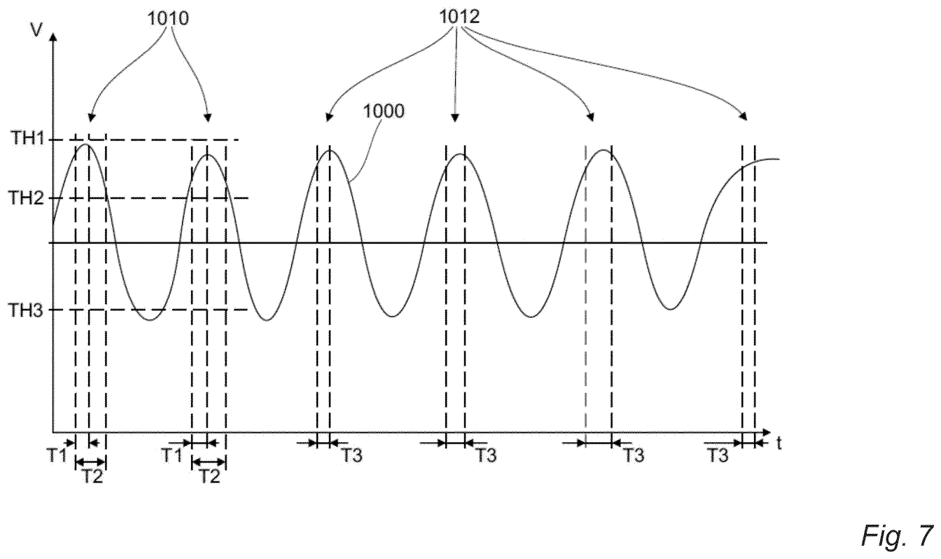

FIG. 7 shows an example of a typical sensor signal 1000 in volts V over the time t, for example generated by an optical sensor like the sensor 215a, 215b (FIGS. 2, 3) and additional readings 1010 taken before the standard readings 1012. The additional readings 1010 may be taken with different parameters than the standard readings, for example over a time span T1 or T2 with T1<T2, while the standard readings may be taken over a time span T3, which may longer or shorter than or equal to T1, T2. The additionally taken readings 1010 may then be processed to determine the sensor condition, particularly they can be compared with one or more thresholds TH1, TH2, TH3.

Threshold TH1 may indicate for example that a reading is too high and probably the sensor signal failed to a supply voltage. Threshold TH2 may indicate that a sensor suffers from degradation when its sensor signal is too close this threshold. Threshold TH3 may indicate that a reading is too small and probably the sensor signal failed to ground.

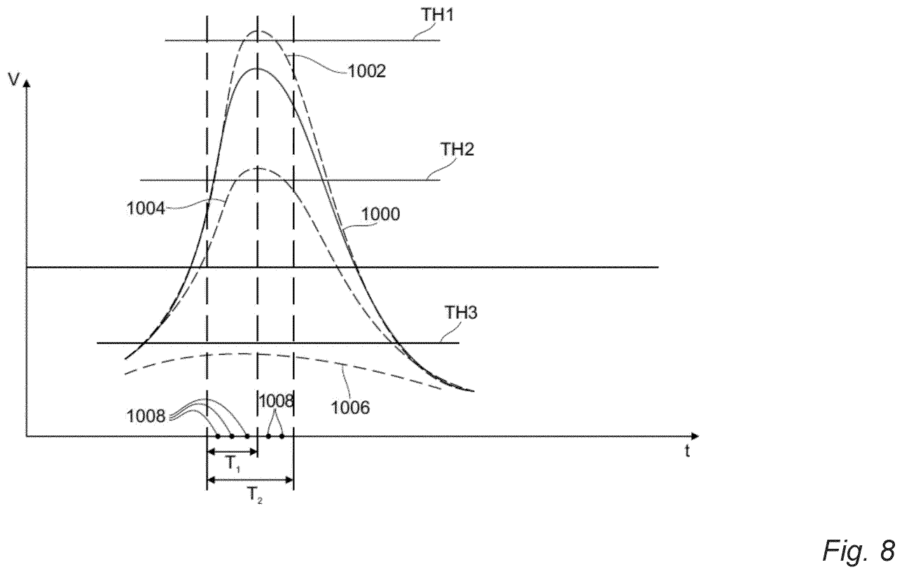

FIG. 8 shows the thresholds TH1-TH3 and examples of sensors signals 1002, 1004, 1006, which could indicate a degradation of the sensor generating the sensor signal. Samples of the sensor signals 1000, 1002, 1004, 1006 can be taken at dedicated sampling times 1008 during certain time spans T1, T2, wherein the time span T2>T1. It should be noted that the taking of samples at times 1008 may vary from the samples taken for standard readings for calculating a delivered dose. Also, the sampling frequency may differ between the additional readings and the standard readings as well as further parameters such as level at which samples are taken. For example, the additional readings can be taken at higher levels than the standard readings, in case of an optical sensor such as the sensor 215a, 215b higher supply voltages and currents may be used for the additional readings. The samples may be sampled signal voltages, and can be averaged to obtain an average sample voltage over the time spans T1, T2 for further processing. It is also possible to process each single sample taken during the time span T1, T2. Instead of several samples, also the signals may be integrated over the time span T1, T2, and the integral may be further processed. Either the samples or any value other value taken within the time span T1, T2 may be regarded as a reading of the sensor signals 1000, 1002, 1004, 1006.

The sensor signal 1000 is a typical output generated by a sensor with a condition sufficient for accurately calculating delivered doses. Signal 1002 exceeds threshold TH1, and, thus, may for example indicate that some circuitry of the sensor outputting this signal may be failed to supply voltage. Signal 1004 is close to threshold TH2 and may indicate a sensor degradation, for example when the sensor is no longer able to output a sensor signal with a higher amplitude (for example, when the LED or photodiode or phototransistor of an optical sensor are degraded). Signal 1006 is below threshold TH3, which may be an indication that some circuitry of the sensor outputting this signal may be failed to ground voltage (for example 0 volts). Threshold TH2 as well as the other thresholds may be also implemented as a range, within which the samples or any value derived from the samples must lie, to indicate a certain condition of the sensor.

In the following, further examples of processing, particularly examining the additionally taken readings are listed:
A long additional reading may be taken before and after a standard measurement period, and if the two values of the long additional readings are not similar, then it may be interpreted that the sensor performance decreased during operation.
A reading taken from a sensor when it is off reads greater than a low threshold value may indicate that the sensor is locked (for example to supply voltage) or has a floating signal.
Readings taken during dispense lie outside of a band determined by a reading taken before dispense.
A reading taken from a sensor when it is at maximum level is below a predetermined threshold may indicate that the sensor is degraded or a floating signal.
The maximum readings taken during dispense do not lie within a certain number of standard deviations away from the average reading taken during dispense.
A long reading taken after the dispense varies outside an allowable tolerance may indicate either a fault or that the system continues to move. This might be compared to some other known value such as the status of an electrical circuit (e.g. a make or break switch).
The maximum reading(s) taken during a dispense is/are higher than a threshold may indicate a failure locking to supply voltage (for example signal 1002 and threshold TH1 in FIG. 8).
The minimum reading(s) taken during a dispense is/are lower than a threshold may indicate a failure locking to ground (for example signal 1006 and threshold TH3 in FIG. 8).
Reading taken too close to a threshold indicate sensor degradation (for example signal 1004 and threshold TH2 in FIG. 8).

The result(s) of the analysis of the additional readings, particularly the determined condition of the sensor such as for example degraded, faulty of the like, can then be stored with a dose record, or as an individual record itself, for example in the main memory 24 of the sensor unit 700 shown in FIG. 6. This information can be communicated via the communication unit 27 to an external computing device, which may include a smartphone, or directly to a user via the display unit 30, or stored in the main memory 24 for later analysis by the user, a healthcare professional and/or the manufacturer. For instance, if it is determined that there is a fault condition of the sensor, the user may be recommended to stop using the drug delivery device or add-on device and replace with a new device, for example by alerting the user with a visual, tactile, and/or audible alert. The alert can be for example displayed on the display unit 30, signalled to a user by a vibration of the drug delivery device generated with a vibration motor comprised by the drug delivery device or an external computing device connected to the drug delivery (add-on) device, and/or signalled by generating a buzzer sound via buzzer integrated in the drug delivery device or a loudspeaker of an external computing device connected with the drug delivery (add-on) device. The dose record may also be flagged as being potentially inaccurate to the user when a non-sufficient sensor condition was determined.

Next, a specific embodiment in an injection device with connectivity is described.

The device has two optical sensors of an opto-coupler type (with emitter and detector) A and B (similar to the system shown in FIGS. 2, 3, A and B correspond to sensors 215a and 215b). In order to detect the effects of failures additional sensor readings are taken at the end of a dose event. Taking the readings at the end of the dose is chosen because in this condition the sensors should be either reading close to their minimum or close to their maximum and are therefore more predictable (because they should be pointing at black or white reflectors and not close to an edge between white and black).

These specific checks are seeking to find sensors that read “high” when they should not do, are frozen in value and do not respond to changes in light, read “low” when they should not do and are degraded such that they read somewhere too close to the threshold (where the threshold may be a software used threshold to digitise the signals).

The specific checking method may be as follows:

Aₙₒᵣₘ and Bₙₒᵣₘ are the two last standard readings that are read at the end of a dose event after a switch has opened again and the debounce period is finished.

Two additional special readings are taken after the normal readings: A pause of 250 μs is allowed, then a reading is taken from each sensor A and B with a long sensor LED excitation (for example 4 to 6 times longer than a normal pulse). These special readings are denoted as A_{bright} and B_{bright}.

Then a further pause of 1 ms is allowed (for charge dissipation), then a sensor reading is taken from each sensor A and B with no excitation of the sensor LED. These readings are denoted as A_{off} and B_{off}.

An error code is generated if any of the following conditions are true (where A_{threshold} and B_{threshold} are predetermined custom transition threshold values between black and white, the specific values are only examples, and may differ in practice):

| Condition | Error code |
|---|---|
| 0.5 * A_{threshold} < Aₙₒᵣₘ < | sensor degraded, or floating |
| 1.25 * A_{threshold} | signal |
| 0.5 * B_{threshold} < Bₙₒᵣₘ < | sensor degraded, or floating |
| 1.25 * B_{threshold} | signal |
| A_{bright} < A_{off} + 50 | ADC, or sensor locked to any |
| | value |
| B_{bright} < B_{off} + 50 | ADC, or sensor locked to any |
| | value |
| A_{off} > 100 | floating signal - also locked to |
| | supply |
| B_{off} > 100 | floating signal - also locked to |
| | supply |

The above mentioned specific values may not be construed as limiting the scope of the invention, and multiple different configurations could be chosen to embody the same invention.

This specific embodiment of the method for checking sensors of the injection device may be suitable to improve the application of the injection device since dose measurements may be made with a higher accuracy particularly over the lifetime of the injection device.

An accurate repeatable performance over the lifetime of a sensor of a drug delivery device or of a drug delivery add-on device may ensure that information such as dose records of an injection system can be correctly recorded. The herein disclosed checking of a sensor may be particularly performed as self-checking to identify one or more failures, errors and/or faults in a system employing the sensor. Moreover, the self-checking could be extended to monitor performance of the sensor over time, and account for degradation for example due to ageing or the presence of external contaminants e.g. water or dust.

As herein disclosed, particularly single fault conditions can be detected by checking a sensor. Such detectable single fault conditions could include, but not limited to, broken components, a broken track, the shorting of tracks to other tracks, ageing, or the presence of water/debris/dust degrading performance. These could lead to faults including, but not limited to, pulling a sensor reading to ground voltage, pulling a sensor reading to supply voltage, creating electrically ‘floating’ readings, locking readings i.e. readings that do not change when exposed to stimulus.

It should be noted that embodiments may be provided and configured for checking not only the above described optical sensors, but in principle any kind of analogue sensor employable in drug delivery devices and drug delivery add-on devices, for example accelerometers, light sensors, sound sensors, pressure sensors, temperature sensors, proximity sensors, infrared sensors, ultrasonic sensors, colour sensors, humidity sensors, tilt sensors, flow sensors, magnetic/Hall effect sensor, radiation sensors, lidar, electrical current sensors, optical sensors, force/torque sensors, strain gauges.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N—(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′) 2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding.

Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1. A method for checking a sensor (215a, 215b) of a drug delivery device (1) or of a drug delivery add-on device, wherein the sensor (215a, 215b) is provided and configured to detect expelling of a drug dose being delivered with the drug delivery device (1) and to output a respective sensor signal (1000), and wherein the method comprises
taking readings (1010) of the sensor signal in addition to readings (1012) taken for calculating the dose delivered with the drug delivery device (1), and
processing the additionally taken readings (1010) for determining at least one condition of the sensor.

2. The method of claim 1, wherein the additional readings (1010) are taken before, after, at the start of, at the end of, and/or during the delivery of a drug dose with the drug delivery device (1), and/or the additional readings (1010) are taken over the entirety, a portion, or multiple portions of the delivery of a drug dose with the drug delivery device (1).

3. The method of claim 1 or 2, wherein the additional readings (1010) are taken with the same parameters as the readings (1012) for calculating the delivered dose.

4. The method of claim 1 or 2, wherein the additional readings (1010) are taken with different parameters than the readings (1012) for calculating the delivered dose, particularly the additional readings (1010) are taken for a longer or a shorter time, at a higher or lower level and/or frequency than the time, frequency or level for or at which the readings (1012) for calculating the delivered dose are taken.

5. The method of any preceding claims, wherein at least one period of not taking readings is included between the taking of the readings (1012) for calculating the delivered dose and the taking of the additional readings (1010).

6. The method of any preceding claim, wherein the processing of the additionally taken readings (1010) for determining a condition of the sensor (215a, 215b) comprises examining the additionally taken readings (1010) against at least one threshold (TH1, TH2, TH3) and determining the at least one condition of the sensor (215a, 215b) depending on the examination.

7. The method of claim 6, further comprising examining the readings (1012) taken for calculating the delivered dose against the at least one threshold (TH1, TH2, TH3) and using this further examination for determining the at least one condition of the sensor (215a, 215b).

8. The method of any preceding claim, further comprising storing the at least one determined condition of the sensor (215a, 215b) in a dataset together with the calculated delivered dose, or storing the at least one determined condition of the sensor (215a, 215b) in a dataset separate from the calculated delivered dose.

9. The method of any preceding claim, further comprising generating an error signal when the at least one determined condition of the sensor (215a, 215b) does not fulfil one or more predefined conditions.

10. The method of claim 9, further comprising outputting an alert informing of the non-fulfilment of the one or more predefined conditions by the at least one determined condition of the sensor (215a, 215b).

11. The method of any preceding claim, wherein the sensor (215a, 215b) comprises an optical sensor having a light emitter and light receiver and being provided and configured for detecting transitions between different regions (70a, 70b) of a moving component of the drug delivery device (1) and to output a signal as the sensor signal (1000) comprising the detected transitions, wherein the optical sensor is controlled with parameters for taking the additional readings (1010) 30 differing from the parameter for taking the readings (1012) for calculating the dose delivered with the drug delivery device (1).

12. A device for controlling a sensor (215a, 215b) of a drug delivery device (1) or a drug delivery add-on device, the device being configured to implement a method of any preceding claim, the device particularly comprising a controller (23), particularly a microcontroller, the controller being configured by a program to implement the method of any preceding claim.

13. The device of claim 10, further comprising one or more of the following: a storage unit (24) for storing calculated delivered doses and/or at least one determined condition of the sensor; a communication unit (27) configured for communicating with an external computing device; a user interface (31) configured for receiving user inputs for configuring the device and/or for outputting information about delivered doses and/or at least one determined condition of the sensor; a display unit (30) configured for displaying information about delivered doses and/or at least one determined condition of the sensor.

14. A sensor unit of a drug delivery device (1) or of a drug delivery add-on device, the sensor unit comprising a sensor (215a, 215b) controlled by a device of claim 12 or 13, wherein the sensor unit is provided and configured for integration in a drug delivery device (1) or a drug delivery add-on device.

15. A drug delivery device (1) or a drug delivery add-on device, particularly an injection pen, comprising a sensor unit of claim 14.
